(19) United States
(12) Patent Application Publication
(10) Pub. No.: US 2023/0213505 A1
(43) Pub. Date: Jul. 6, 2023

(54) A PROCESS FOR APPLYING A COATING COMPRISING ONE OR MORE POLYSACCHARIDES WITH BINDING AFFINITY FOR BIOANALYTES ONTO THE SURFACE OF A MEDICAL SAMPLING DEVICE, AND THE MEDICAL SAMPLING DEVICE FOR CAPTURE OF BIOANALYTES PROVIDED WITH THE COATING

(71) Applicant: IDRIS ONCOLOGY B.V., (US)

(72) Inventors: Peter Immanuël WIEGMAN, Leiden (NL); Hans Peter MULDER, Leiden (NL)

(21) Appl. No.: 18/021,159

(22) PCT Filed: Aug. 9, 2021
(86) PCT No.: PCT/EP2021/072169

§ 371 Date: Feb. 13, 2023

(30) Foreign Application Priority Data
Aug. 14, 2020 (NL) 2026271

(51) Int. Cl. G01N 33/543; G01N 33/50

(52) U.S. Cl. CPC G01N 33/54353; G01N 33/5091

(57) ABSTRACT

The present invention provides a coating comprising one or more polysaccharides with binding affinity for bioanalytes, for application on the surface of a medical sampling device, wherein the one or more polysaccharides are end-point attached to the surface of the medical device, and wherein the one or more end-point attached polysaccharides have one or more polysaccharides end-point grafted to the side groups extending from their backbone. The invention also provides a process for preparing the same as well as a medical diagnostic device comprising the coating.

### TECHNICAL FIELD

This invention concerns a coating for capture of bioanalytes comprising one or more polysaccharides that are end-point attached to the surface of a medical sampling device. Moreover, this invention concerns a medical sampling device, provided with the new coating resulting in the enhanced capture of bioanalytes, and of circulating tumor cells (CTCs) in particular, which can then be released for analysis and diagnosis.

### BACKGROUND ART

Devices are known for the capture of bioanalytes wherein the object to be analyzed is bioactive and is selected from the group comprising macromolecules, polynucleotides, RNA, DNA, proteins, marker proteins, lipoproteins, polypeptides, antibodies, autoantibodies, hormones, antigens, cells, CD44+ cells, viruses, bacterial cells, parasites, fungus cells, tumor cells, stem cells and/or cells that originate from a fetus during pregnancy, or parts thereof. Of particular interest are devices for the capture of circulating tumor cells.

Chemotherapies have become much more advanced over the past two decades. Whereas older ‘generic’ chemotherapies simply kill all cells in the body that are growing quickly (resulting in damage to healthy tissues), modern targeted chemotherapies are designed to only affect specific (cancer) cells and minimize collateral damage. Although targeted chemotherapies have provided a major improvement in cancer care, their true potential remains unrealized. Because cancer cells are continuously mutating, they can adapt and become resistant against a targeted chemotherapy. This ultimately results in the targeted chemotherapy becoming ineffective. Considering that cancer is often fatal and targeted chemotherapies are expensive, there is a great need for tools that allow for the tracking of when and how the tumor cells have become resistant, so that the therapy can be adjusted accordingly.

Currently the most widely accepted way to track tumor mutations is to take a biopsy, a direct tissue sample of the tumor. This is an invasive procedure, which is inherently painful, carries a risk of locally spreading the disease, and even when performed well only gives a reflection of the precise location sampled at that particular time. Due to patient considerations successive biopsies of all tumors are rarely performed. Liquid biopsies sample the blood instead of the tumor, and are the most promising tools for continuously tracking tumor mutations because of their relative ease and safety. There are many different liquid biopsy approaches, including the isolation of circulating tumor cells (CTCs), tumor derived vesicles (e.g. exosomes) and circulating tumor DNA (ctDNA). Of all these approaches, CTCs provide the most comprehensive and in depth information on tumor resistance, because they contain a complete profile of DNA, RNA and proteins. However, CTCs are extremely rare in the blood: typical blood samples average less than 1 CTC per milliliter of blood, among billions of other cells. Many more cells (100+) are needed to track tumor resistance reliably, which is why there is a need for technologies that can efficiently isolate CTCs from the blood. The present invention therefore concerns the selective capture of CTCs.

Of similar interest are medical sampling devises to diagnose sepsis. Sepsis is an inflammatory immune response triggered by an infection. Bacterial infections are the most common cause, but fungal, viral, and protozoan infections can also lead to sepsis. Sepsis guidelines recommend obtaining blood cultures as quickly as possible to obtain an accurate diagnosis. However, blood cultures can take up to several days, while sepsis is an acutely dangerous condition and treatment should be commenced as soon as possible. Being able to quickly and selectively analyze the source of the infection would therefore be very beneficial.

The application of optical fiber, catheter or wire-based devices, which are decorated with biologically active molecules, for diagnostic tasks such as the detection and capturing of DNA, proteins, cells, and others from biological samples or even from living organisms is known.

WO 2006131400 teaches the decoration of a stainless steel wire with metallic islands that are modified with antibodies for specific cell capture. The metallic islands with sizes in the 100 nm regime were fabricated by using a sphere-monolayer as a shadow mask during the deposition step of a gold layer. The gold islands were modified with thiolated linker molecules that bind specific antibodies.

In EP1907848 a diagnostic-nanosensor is described, e.g., in the form of a catheter or spring wire, consisting of a carrier comprising areas on two-dimensioned arched metallic nanostructures with detection-molecules. The diagnostic nanosensor may be used for the direct detection and isolation of rare molecules or cells out of the peripheral blood or the body. This application technique enables diagnostic procedures that were not possible before: prenatal diagnoses of chromosomal aberration using fetal trophoblasts present in maternal circulation; cancer diagnoses and monitoring of cancer therapy based on the detection of disseminated cancer cells in the body.

EP2344021 relates to a device for detecting analytes, comprising a polymer fiber and capturing molecules, wherein the capturing molecules bind to an analyte and/or a linker molecule. The capturing molecules are selected from the group comprising antibodies, antigens, receptors, polynucleotides, DNA probes, RNA probes, polypeptides, proteins and/or cells. The functionalized polymeric fiber described in this document as having microstructures or surface geometries on the surface can be introduced into a biological sample, such as a blood sample or into a vein of a living organism. During a period of at least a few seconds and several hours, the fiber collects the respective target analyte through its biofunctional coating. After the collection process has finished, the fiber is retracted and the captured material is separated from the fiber for analysis.

Likewise from EP2547250 a biodetector is known with a functionalized surface for isolating molecules or cells from the human body. This biodetector is introduced into the human body for the isolation and enrichment of target molecules and target cells and after a short period is once again removed from the human body.

In “An ensemble of aptamers and antibodies for multivalent capture of cancer cells”, in Chem. Commun., 2014, 50, 6722, by Jinling Zhang et al, an optimized ensemble may be found that functions as a multivalent adhesive domain for the capture and isolation of cancer cells. The ensemble may be incorporated into a microfluidic device that contains micro-pillar arrays. The antibodies and aptamers were immobilized onto the microfluidic channels using avidin and biotin reactions as described in “Aptamer-enabled Efficient Isolation of Cancer Cells from Whole Blood Using a Microfluidic Device” by Sheng W, Chen T, Katnath R, Xiong X L, Tan W H, Fan Z H. Anal. Chem. 2012; 84:4199-4206. The microfluidic device is reported to yield a capture efficiency of >95% with a purity of ˜81% at a flow rate of 600 nL/s. This demonstrates technical feasibility, but the flow rate is insufficient to be clinically applicable. More precisely, at this rate one would need more than 46 hours of continuous operation at 100% efficiency to get a hypothetical yield of 100 CTCs, which is not practical at all.

Tumor cells are currently isolated in vitro with high sensitivity and selectivity for subsequent screening of clinically relevant parameters. However, given the very low occurrence of CTCs in blood, even the largest blood sample that can be taken still gives very little information. Further improving the sensitivity and selectivity will allow instead to do so in vivo, which will greatly help further screening of clinically relevant parameters. Similar considerations apply in respect of sepsis.

The capture of bioanalytes, such as circulating tumor cells (CTCs), directly from within the bloodstream in a human vein is challenging. As indicated above, capturing bioanalytes is typically done with antibodies, which are highly selective and provide a strong and near permanent bond. However, antibodies are only able to form these bonds at very low relative speeds and they rapidly lose their capability to capture bioanalytes at speeds above 1 mm/s, whereas the speed within the bloodstream is multiple orders of magnitude higher still. Moreover, a problem in the state of art is that trapped bioanalytes, such as CTCs, are often damaged or destroyed while retracting the device after capture or upon release. As a result, detection or diagnosis is no longer possible or trustworthy.

Accordingly, there remains a need for a medical sampling device with improved sensitivity and selectivity to isolate bioanalytes such as CTC's from a circulating system, preferably from the circulatory system, more preferably directly in the bloodstream, i.e. a modified guidewire or catheter with increased capture rate that allows for release for further screening of clinically relevant parameters.

In a paper by Zhi Sheng-liang et al, “Fabrication of Carbohydrate Microarrays on Gold surfaces: Direct Attachment of Nonderivatized Oligosaccharides to Hydrazide-modified Self-Assembled Monolayers”, Analytical chemistry, part 78, No. 14, 2006, pp. 4788-4793, a process is disclosed for applying a coating comprising heparin on the surface of a microarray, wherein the heparin is end-point attached to amine groups on its surface. The microarray can be used for mapping carbohydrate-protein recognition events.

WO2010019189 describes inter alia a medical device having a surface which comprises a coating layer based on end-point attached heparin, which is covalently attached to said surface through a link comprising a 1,2,3-triazole. The heparin is used as an anti-coagulant compound. The medical device is not used as a sampling device. Although branching is mentioned this is neither of the heparin, nor is it mentioned to improve the capture rate.

In WO2013188073, a method is described for the removal of mediators that contribute to the pathogenesis of cancer from blood by contacting the blood outside of the body with a solid. This is therefore not a diagnostic device. The method is specifically described for heparin as other carbohydrate surfaces may be significantly less blood compatible than heparinized surfaces and can lead to increased thrombogenicity. On the other hand, having a coating that would further improve the efficiency of the removal of such mediators would be of particular interest.

The main application of this invention is to improve upon existing methods to isolate bioanalytes such as CTCs by providing a coating that functions as an additional selection mechanism and increasing the sensitivity of existing selection mechanisms while also imparting hemocompatibility. The coating can be applied in in-vivo enrichment tools such as modified guidewires and catheters that capture CTCs directly in the bloodstream (e.g. Gilupi CellCollector®). However, the coating may also be applied in in-vitro technologies such as magnetic beads separation (e.g., The CELLSEARCH® Circulating Tumor Cell Kit, which is intended for the enumeration of CTCs of epithelial origin (CD45−, EpCAM+, and cytokeratins 8, 18+, and/or 19+) in whole blood) and some microfluidic flow cells as well.

### SUMMARY OF THE INVENTION

The present invention provides a coating comprising one or more polysaccharides with binding affinity for bioanalytes, for application on the surface of a medical sampling device, wherein the one or more polysaccharides are end-point attached to the surface of the medical device, and wherein the one or more end-point attached polysaccharides have one or more polysaccharides end-point grafted to the side group extending from their backbone. The invention further provides a process for applying the coating, comprising a step (A) of functionalizing the surface, followed by a step (B) of end-point attaching the one or more polysaccharides to the functional groups on the surface, followed by a step (C) of end-point attaching one or more polysaccharides onto the polysaccharides that are end-point attached to the functional groups on the surface. Preferably, the step of end-point attaching the one or more polysaccharides to the functional groups on the surface is followed with a step (D) of blocking any residual functional groups on the surface. The order of steps (C) and (D) may be reversed. The present invention also provides the medical sampling device for capture of bioanalytes such as circulating tumor cells having the coating applied thereon.

The present invention also provides a method for the capture of bioanalytes such as circulating tumor cells and a method for release and analysis of the captured bioanalytes.

### DRAWINGS

FIG. 1, a schematic presentation of the application of the prior art coating on a substrate applied on the surface of a medical sampling device, based on polysaccharides that are not end-group attached.

FIG. 2, a schematic presentation of the application of the present coating on the substrate, based on polysaccharides that are end-group attached, with the active groups on the surface of the substrate being blocked.

FIG. 3, a schematic presentation of the application of the present coating on the substrate, wherein receptors and/or ligands are grafting onto the one or more polysaccharides.

FIG. 4, a histogram illustrating the effect of the coating on the cell velocity and on the capture rate.

### DETAILED DESCRIPTION OF THE INVENTION

The medical sampling device according to the present invention has an improved sensitivity and selectivity to isolate bioanalytes and in particular CTC's directly in the bloodstream. It may be in the form of a guidewire or catheter. Importantly, it shows an increased capture rate whilst at the same time allowing for release of the captured bioanalytes for further screening of clinically relevant parameters. In addition, the coating may be applied on a solid for the removal of mediators that contribute to the pathogenesis of cancer from blood by contacting the blood with the coated solid.

The process of the present invention starts with the functionalization of the surface of the medical sampling device. This is done preferably by amination, i.e., by introducing a free amino group on the surface.

Step (A) of the present process preferably comprises the amination of the surface of the medical sampling device. Preferably, the amination is performed with a diamine. Next, a compound acting as a linker is attached, which is preferably a dialdehyde, more preferably glutaraldehyde.

Step (B) of the present process comprises the end-point attachment of a polysaccharide to the linker that has reacted with the amine group. Preferably hyaluronic acid is used as polysaccharide, onto which a diamine is attached, by way of reductive amination to its terminal end. The reductive amination may be carried out in the presence of a reducing agent. It is preferably carried out with adipic dihydrazide in the presence of sodium cyanoborohydride.

Step (C) of the present process comprises the grafting, i.e., end-point attachment of a polysaccharide onto the backbone of a polysaccharide that is attached to the surface of the medical sampling device. In this case hyaluronic acid is used in step (B) and in step (C). This is done by carbodiimide coupling, although other reactions are possible. This process may be repeated twice or more often, to create layers of carbohydrates grafted on carbohydrates, grafted on carbohydrates. These polysaccharides may be identical, belong to the same class but differ in molecular weight, or be unrelated.

End-point attachment of polysaccharides, however, leaves unreacted amino and aldehyde groups from the linker on the surface of the medical sampling device. These residual functional groups will negatively affect the selectivity of the applied coating and therefore need to be blocked. Preferably, these residual functional groups are blocked in step (D) under mild conditions, without affecting the end-group attached carbohydrates. For instance, residual amino groups may be converted into aldehyde groups, and these residual aldehyde groups may be blocked by reaction with an amino acid, thereby creating a free acidic group that no longer affects the selectivity of the applied coating. For stability reasons, the imine bonds may be reduced, preferably with the use of sodium cyanoborohydride. The order of step (C) and (D) may be reversed.

In the present invention, polysaccharides with binding affinity for bioanalytes are used. Polysaccharides with binding affinity for bioanalytes are well known in the art. The affinity may be inherent and e.g. result from detection receptors on the surface of the bioanalytes, but may also be the result of detection receptors attached onto the polysaccharides. Within the scope of this patent application, polysaccharides with binding affinity for bioanalytes such as CTC's form at least a temporal link with the bioanalytes either through interaction of the receptors on the bioanalytes with the polysaccharide itself and/or through interaction of the detection receptors attached onto the polysaccharide. The interaction may be through the polysaccharides that are end-point attached to the surface of the substrate, through the polysaccharides that are grafted onto the aforementioned polysaccharides or a combination of both.

The polysaccharides used in the coating of the present invention are long chain polymeric carbohydrates composed of monosaccharide units bound together by glycosidic linkages. Examples include storage polysaccharides such as starch, glycogen and galactogen and structural polysaccharides such as cellulose and chitin. Preferably the polysaccharides are heterogeneous, more preferably hemocompatible glycosaminoglycans, more preferably glycosaminoglycans that are native to the human body. It may be an oligosaccharide consisting of only a few repeating sugar units, as well as a long polysaccharide exceeding a million Daltons in molecular weight, as well as mixtures thereof. Typically it has from 40 to 3000 monosaccharide s as repeating units in the polymer backbone.

By using end-point attachment, and due to the negatively charged side groups commonly found in polysaccharides, the polysaccharide molecules repel each other and may therefore project away from the surface of the medical sampling device, extending farther into the blood, thereby maximizing its availability for binding specific receptors on tumor cells. This principle applies equally to the polysaccharides that are grafted onto polysaccharides. Typically, the coating has a thickness in the range of 0.1 μm to 2 μm.

Most preferably, the coating of the present invention comprises or even entirely consists of hyaluronic acid (HA), a hemocompatible glycosaminoglycan that is native to the human body. HA is therefore preferably used to be end-group attached to the surface of the medical sampling device, but also to be end-group attached to the polysaccharide that is end-group attached to the surface of the medical sampling device. HA is preferred for several reasons, as discussed hereafter.

Due to its carboxylic acid side group, HA can be easily chemically modified and coated onto surfaces. Moreover, the side groups of HA allow for facile coupling of additional molecules such as antibodies and other receptors.

HA is of particular interest, as it has been discovered that tumor cells often show abundant expression of hyaladherins such as CD44, receptors that specifically bind HA. Most tumor cells can therefore adhere to a coating of HA, whereas HA is otherwise generally repellant and non-fouling. By using end-point attachment, HA is applied such that it is maximally available for binding specific receptors on certain types of cells, most notably tumor cells.

The interaction between CD44 and HA is not limited to simple cell adhesion. The present inventors have demonstrated that CD44-positive cells can roll over HA coated substrates. This interaction might play a role in the extravasation and homing of immune cells, and might therefore also be involved in CTC extravasation and metastasis. The ability of HA-coated surfaces to induce cell rolling has been found to be very beneficial in the enrichment of CTCs, because the rolling action likely reduces the speed. This is beneficial because; the higher the flow velocity of a CTC along the surface of a coating, the lower the chance of binding the cell to the coating. In other words, besides the ability of tumor cells to specifically adhere to HA-coated surfaces, tumor cells in flow may also be more easily captured due to its rolling action.

Conveniently, HA can be produced by bacterial fermentation, which avoids the potential toxins and pathogens of animal-derived HA. Bacterial fermentation has enabled industrial production of HA, as evidenced by numerous clinical and cosmetic products, and even dietary supplements. Preferably, the HA has a molecular weight in the range of 40 kDa to 2 MDa, preferably in the range from 50 kDa to 1.5 MDa. The HA may also be substituted, wherein at least some of the functional groups along the polymer backbone have been substituted with other functional groups.

Importantly, HA can be specifically degraded by enzymes under mild conditions thereby allowing controlled release of the CTCs. For instance, HA can be selectively degraded under mild conditions using hyaluronidases, which has also seen clinical applications. In summary, HA is a versatile molecule with many applications in cancer therapy and diagnosis. As a coating, it can provide a unique combination of selective tumor cell adhesion, hemocompatibility and rich chemistry. In other words, the HA coating is optimized for the capture of circulating tumor cells from whole blood, both in-vivo and in-vitro.

As mentioned above, the binding affinity of the polysaccharides in the coating may be created or enhanced by adding receptors and/or ligands for CTC's thereon, such as antibodies, preferably monoclonal antibodies, chimeric antibodies, humanized antibodies, antibody fragments or amino acid structures and amino acid sequences, nucleic acid structures or nucleic acid sequences, and the like. For instance, the interaction of HA and aptamers and antibodies is such that the end-point attached HA, the aptamers and the antibodies together are believed to slow down the passing CTC's and therefore increase the selective capture.

As mentioned above, the coating of the present invention is preferably provided by first introducing an amine group on the surface of the sampling device, followed by a linker and then end-point attaching the polysaccharide, preferably HA. Amination can be achieved best on a medical sampling device having a polymeric surface. This can be accomplished, for example, by the aminolysis of ester-containing polymers such as polyurethanes (PU), polyesters (e.g. PET) or polymers containing esters in their side group (e.g. PMMA). In principle any polymer capable of aminolysis may be used. Surface amination can also be achieved through silanization, or ammonia-based plasma treatment. Aminolysis is preferably carried out with a diamine, more preferably with ethylenediamine or hexamethylene diamine.

Next a linker is attached to the amine group on the surface. Preferably a dialdehyde is used, as it allows the end-point attachment of the end-aminated polysaccharide under very mild conditions. However, It is also possible to use, e.g., citrate/diamine-citrate, a tri-carboxylic acid, to conjugate to a carboxylic acid-coated surface through a diamine intermediate. If each citrate binds to the aminated surface through a single one of its carboxylic acids, for each amine group exposed on the surface there will be 2 carboxyl groups. This will effectively double the number of carboxyl groups on the surface and therefore the grafting density.

The polysaccharide may be end-point attached directly to the linker, or the terminal end of the polysaccharide may be modified to allow reaction with the linker that is attached to the aminated surface. For instance, the terminal end of the polysaccharide and/or the surface amine groups may be modified with a thiol group.

The previous steps have resulted in a substrate with the polysaccharide end-attached to the surface. However, the end-point attachment reaction is unlikely to couple a polysaccharide molecule to every available linker on the surface. Consequently, there will be active groups on the surface, e.g., free aldehyde groups, which is disadvantageous for two reasons: the residual functional groups from the linker may provide non-specific adhesion sites, and the residual functional groups may cause the polysaccharide that is end-point attached to flatten on the substrate. Hence, the residual surface functional groups are preferably blocked with a functional group that prevents interactions between the substrate and the polysaccharide. The blocking of residual surface functional groups can also prevent undesirable chemical reactions between the substrate and other reactants in downstream reactions, such as the coupling of detection receptors. As mentioned above, if the surface amine has reacted with a dialdehyde, this is preferably achieved with an amino acid, because this reaction is simple, quick and does not affect the end-point attached polysaccharide. The dialdehyde can be any molecule with two or more aldehyde groups, but glutaraldehyde is preferred because it is readily available and reacts efficiently. The dialdehyde will decorate the substrate with unreacted aldehydes, which can then be reacted with any amino acid based on the desired properties. Preferably 6-aminocaproic acid is used, because it does not have any side groups. Other good alternatives would be aspartic acid and glutamic acid, as these amino acids contain an additional carboxy moiety, which is advantageous for creating additional sites for attaching polysaccharides or (discussed hereafter) for attaching receptors.

Next, the coating is further modified by grafting additional polysaccharides onto the polysaccharides end-point attached to the surface. As mentioned before, this is preferably the same hyaluronic acid, with the same molecular weight.

The end-point attached polysaccharides will have a ‘brush’-like architecture that can be made into a ‘bottlebrush’-like structure by coupling polysaccharides to the side groups of the end-point attached polysaccharide, thereby creating a branched structure. Various coupling reactions may be used. Coupling end-aminated carbodiimide to the end-pointed attached polysaccharide with a carbodiimide coupling reaction is preferred. This approach is preferred because the end-aminated polysaccharide has also been used in previous reactions, and because the carbodiimide coupling reaction is a two-step reaction that allows for control over the maximal number of branching points per bottlebrush-like structure. Preferably, this is done in a two-step approach to ensure that the polysaccharide that is used for grafting can only couple to the end-point attached polysaccharide, and not to itself. For instance, activating the carboxy group of the end-point attached polysaccharide with a good leaving group, such as N-hydroxysuccinimide (NHS) or N-hydroxysulfosuccinimide (sulfo-NHS), may help. The grafting procedure may be repeated to graft polysaccharides on already grafted polysaccharides, thereby increasing the size of the bottlebrush and creating a thicker and denser coating. This may significantly affect the ability of the coating to interact with bioanalytes.

The medical sampling device according to the present invention may therefore be made by applying first a polymeric substrate thereon, which is then functionalized, followed by the end-point attachment of the polysaccharide, preferably HA, whereupon any residual functional groups on the surface are blocked. The end-point attached polysaccharides are then grafted, creating a branched structure (herein referred to as Bottle Brush). Optionally the polysaccharide, preferably HA, is then further modified to add further detection receptors thereon.

For instance, the carboxy groups of HA are easily decorated with detection receptors if the detection receptors have amino groups, which is commonly the case with antibodies and other proteins. Aptamers, if necessary, can be functionalized with an amino group. Decorated HA with detection receptors with available amino groups is preferable because simple and effective carbodiimide coupling can be used. Other non-carbodiimide coupling methods may be used to decorate HA with detection receptors, either with or without intermediate linkers and depending on the moieties available on the detection receptor.

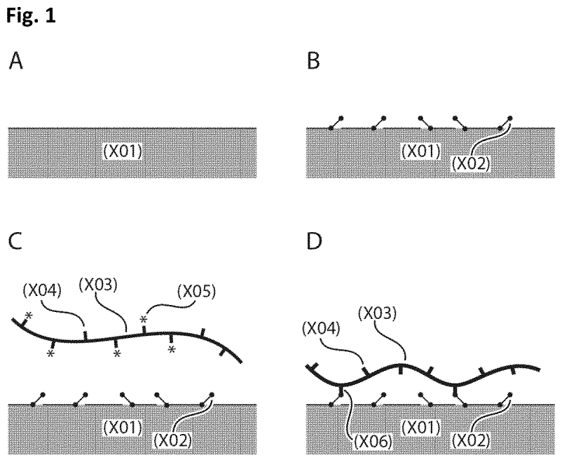

In FIG. 1 (A-D) an artist impression is provided of a coating wherein the polysaccharide X03 is applied onto a polymeric substrate X01 without end-group attachment. In this prior art process, amine groups X02 are provided onto the substrate. The polysaccharide side-groups X04 are activated, creating X05 which react with the amine groups X02, forming connections X06 whereby the polysaccharide is coupled to the amino group of the substrate surface. This is colloquially referred to as “spaghetti”.

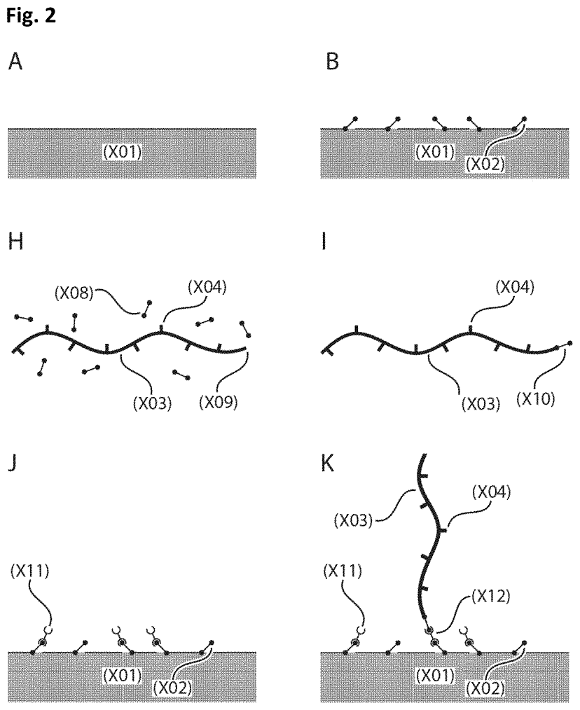

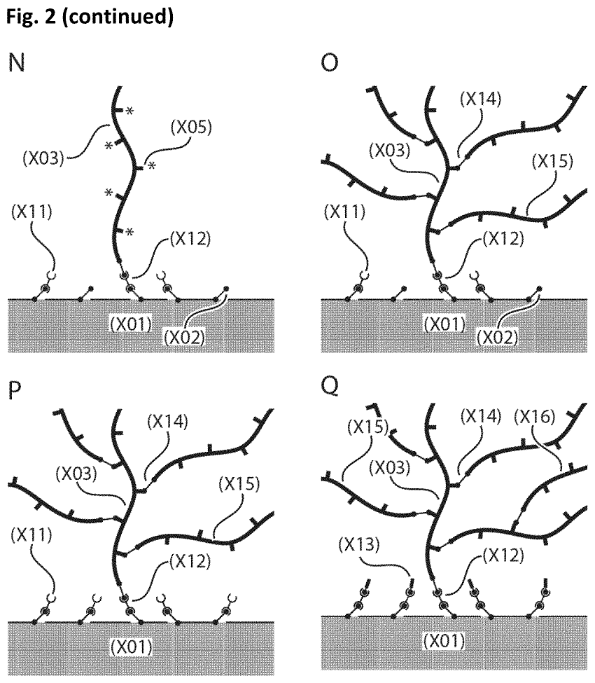

Turning to FIG. 2, a series of steps are provided to apply the coating of the present invention onto the polymeric substrate X01. Again, amine groups X02 are provided onto the substrate X01 (FIG. 2A-B). Meanwhile (FIG. 2H-I), the polysaccharide X03 is reacted at its end X09 with a diamine X08, preferably dihydrazide, resulting in an end-group X10 that facilitates the end-group attachment. In FIG. 2J the substrate is reacted with a dialdehyde to form an aldehyde group X11 on its surface. In FIG. 2K, the end-group X10 is connected with the aldehyde group X11, forming the connection X12. In FIG. 2N, the side groups of the end-attached polysaccharide are activated, X05, for subsequent coupling with X10, resulting in bond X14 in FIG. 2O, through which polysaccharide X15 is end-attached to X03. In FIG. 2P residual amino groups are blocked with dialdehydes, whereas in FIG. 2Q the aldehyde groups are blocked with amino acids.

Finally, by repeating the steps in FIG. 2N-O a polysaccharide X16 is end-attached to X15 in FIG. 2Q.

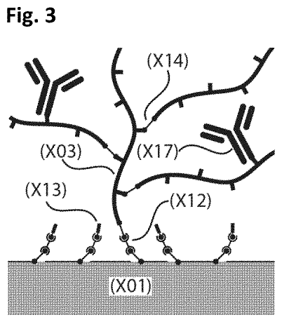

In FIG. 3 receptors X17 are provided onto side-groups X04.

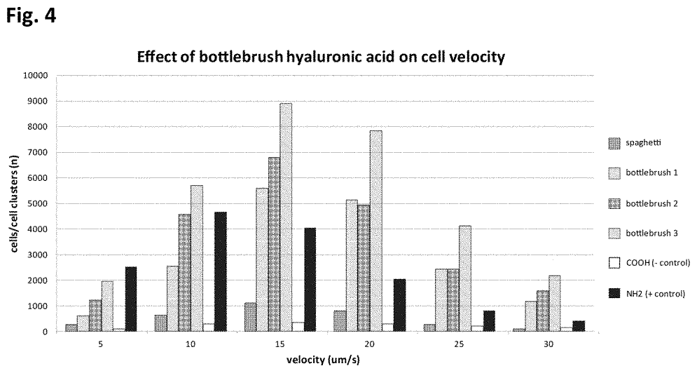

Turning to FIG. 4, a histogram is shown that illustrates the effect of the grafting method on the velocity of cultured breast cancer cells in a flow cell. The histogram contains a positive and negative control, with ‘spaghetti HA’ as baseline. It should be noted that in absence of any interaction between the coating and the cells (−control), the vast majority of cells are flowing too fast to be quantified. Vice versa, cells that interact so strongly with the coating that they adhere, will be counted as the lowest velocity bar (<5 μm/s). Consequently, the higher this bar (at 5 μm/s), the more a coating can capture cells. A detailed discussion is provided in the experimental section below. Whereas bottlebrush 1 is the coating according to the experimental section, with a single grafting step. In bottlebrush 2 and bottlebrush 3 the grafting step of the experimental section is repeated once or twice respectively. Repeating the grafting step results in a higher number of branching points and amount of grafted polysaccharide chains.

The effect of HA on slowing down and adhering cells is illustrated by the following experiments, in which test samples in the form of a coated substrate are tested in a flow cell. The efficiency is compared with two controls: with a COOH coating and an NH₂ coating. It is also compared with a coated substrate where the HA is neither end-point attached nor extending into the flow.

### Examples

Materials used:

Reactants (all purified)

| | |
|---|---|
| EDA | ethylene diamine (E26266 by Sigma) |
| ADH | Adipic dihydrazide (A0638 by Sigma) |
| MQ | purified water (Milli-Q ® Direct 8 Water Purification |
| | System) |
| 96% EtOH | Ethanol with 2.5% IPA (76053184.5000) by Boom BV |
| IPA | 2-propanol |
| Na₂Br₄O₇ | sodium tetraborate (221732) from Sigma |
| NaCl | sodium chloride (793566) from Sigma |
| HCl | hydrochloric acid 37% (124635000) from Arcos Organics |
| NaBH₃CN | sodium cyanoborohydride (156159) from Sigma |
| HA | Hyaluronic acid (50 KDa) from BeautyStuff.nl |
| HA | Hyaluronic acid (1.5 MDa) from BeautyStuff.nl |
| GA | Glutaraldehyde 2.5% (G6257) from Sigma |
| 6AC | 6-aminocaproic acid (A2504) from Sigma |
| PBS | Phosphate buffered saline (P3813) from Sigma |
| MES | 2-(N-Morpholino)ethanesulfonic acid hydrate (M2933) |
| | from Sigma |
| NHS | N-hydroxysuccinimide (130672) from Sigma |
| EDC | N-(3-dimethylaminopropyl)-N′ethylcarbodiimide |
| | hydrochloride (03450) from Sigma |
| VU1D9 | mouse monoclonal antibody (# 5198S) from Cell |
| | Signaling Technology |
| hyaluronidase | hyaluronidase from bovine testes (H3506) from Sigma |
| Detergent | Dreft Original detergent |
| Cells | |
| MCF7 | HTB-22 ™ from American Type Culture Collection, |
| | cultured according to the ATCC protocol; harvested with |
| | Accutase ® and stained with Celltracker ™ |
| | Green CMFDA (C7025) from ThermoFisher Scientific |
| DMEM | Dulbecco's Modified Eagles Medium |
| | (HYCLSH30243.FS from VWR) |
| FBS | Fetal Bovine Serum (F7524) from Sigma |

Substrate

| | |
|---|---|
| 1625PU500001 | polyurethane sheets of shore A90 sold by Technirub |

1. Surface Amination

Aminolysis is a reaction where an amine reacts with an ester to form an amide bond. EDA was used because it reacts efficiently. The protocol described below was used.
a. thoroughly clean the substrate by hand with MQ and detergent
b. ultrasonicate the substrate at 40 kHz in MQ with detergent for 60 minutes at room temperature with the use of a Branson 5510 ultrasonicator
c. wash the substrate with MQ
d. ultrasonicate the substrate at 40 kHz in MQ for 60 minutes at room temperature
e. let the substrate dry
f. prepare 2 M EDA in 96% EtOH
g. incubate substrate in EDA solution for 2 hours (hrs)
h. gently rinse the substrate with 96% EtOH
i. wash the substrate in 96% EtOH for 2+ hrs under light stirring
j. repeat the previous step with fresh 96% EtOH
k. dry for 2+ hrs at 40° C.

An aminated substrate will function as a positive control in the flow setup experiment, as amino groups are a moiety well known for their ability to non-specifically adhere many cells.

2. End Amination of HA (iHA)

The goal of this step is to provide an amino group to the reducing terminal end of the polysaccharide. This is achieved by coupling a diamine to the aldehyde (which is in equilibrium with a lactol) on the reducing terminal end of the polysaccharide, and reducing the resulting bond with a reducing agent. To minimize the coupling of polysaccharide to both amines of the diamine, a molar excess of diamine is used in this reaction. Hyaluronic acid (HA) was used as the polysaccharide. Sodium cyanoborohydride was used as the reducing agent, because it is relatively mild and readily available. Adipic dihydrazide is used in this reaction because the coupling efficiency is high.
a. dissolve 0.1 M Na₂Br₄O₇ and 0.4 M NaCl in MQ
b. adjust the pH to 8.4 with HCl (using a VOS-70005 pH meter)
c. bubble the resulting buffer with N₂ gas for 2+ hrs
d. dissolve 5% w/v HA and a 1000× molar equivalent of ADH in the borate buffer
e. dissolve 0.07 M NaBH₃CN in the volume of borate buffer
f. incubate the borate buffer at 40° C. for 3 days
g. fully dialyze the borate buffer in MQ
   i. use a dialysis membrane or tubing with 10 kDA cutoff
   ii. dialyze in a volume of MQ at least a 100× greater than the borate buffer volume
   iii. refresh the MQ at least 5 times with at least 6 hrs intervals
h. lyophilize the dialyzed solution

3. Glutaraldehyde Coupling

The goal of this step is to make an aminated polymer surface reactive to the end-aminated polysaccharide. This is achieved by coupling a dialdehyde to the surface amino-groups, providing a reactive aldehyde on the surface that can subsequently bind to the amino group of the end-aminated HA. Most dialdehydes should work, but we prefer relatively short and simple dialdehydes like glutaraldehyde because they limit the chance of both aldehydes coupling to the surface.
a. dissolve 2% v/v GA in MQ
b. incubate the polymer surface in the GA solution at room temperature (RT) for 6+ hrs
c. gently agitate frequently
d. gently wash the polymer surface with MQ three times or more

4. End-Aminated Polysaccharide Coupling to the Substrate

The goal of this step is to couple the end-aminated polysaccharide to an aldehyde-functionalized surface, thereby creating end-point attached polysaccharide. The amino-group on the reducing terminal end of the end-aminated polysaccharide will react spontaneously to the surface aldehyde.
a. dissolve 1% w/v of end-aminated MDa HA in MQ
b. incubate the polymer surface with the iHA solution for 6+ hrs
c. gently agitate frequently
d. gently wash the polymer surface with 1× PBS pH=7.4 three times or more

5. Blocking of Residual Surface Groups

The previous steps have resulted in a polyurethane substrate with hyaluronic acid end-attached to the surface. The reductive amination reaction is unlikely to couple a hyaluronic acid molecule to every available surface amine. Consequently, there will be residual surface amines, which is disadvantageous for two reasons: the residual surface amines are a moiety that may provide non-specific adhesion sites, and the positive charge of residual surface amines at physiological pH may cause negatively charged hyaluronic acid that is end-point attached to flatten on the substrate.

The residual surface amines are blocked by reaction with a dialdehyde (GA) and subsequently an amino acid (6AC), because these reactions are simple, quick and do not affect the end-point attached hyaluronic acid.
a. prepare 2% v/v GA in MQ
b. incubate the substrate in the GA solution at RT for 6+ hrs
c. gently agitate frequently
d. gently wash the substrate with MQ three times or more
e. prepare 100 mM 6AC in MQ
f. incubate the substrate in the 6AC solution for 6+ hrs
g. gently agitate frequently
h. gently wash the substrate with 1× PBS pH=7.4 three times or more

6. Grafting of Polysaccharides onto the End-Point Attached Polysaccharide

Grafting is carried out by the following method:
a. dissolve 100 mM NHS and 100 mM EDC in 0.1 M MES buffer pH=5.5
b. incubate the polymer surface with the EDC solution for 1 hrs at RT
c. gently agitate frequently
d. gently wash the substrate with 1× PBS pH=7.4
e. dissolve 1% w/v iHA in 1× PBS pH=7.4
f. incubate the polymer surface with the iHA solution at RT for 5+ hrs
g. gently agitate frequently
h. gently wash the polymer surface with 1× PBS pH=7.4 three times or more

7. (Optional) Decorating of the HA with Detection Receptors

The protocol below describes one method for carbodiimide coupling with the anti-EpCAM antibody VU1D9. There are also other anti-EpCAM antibodies available, but the protocol will most likely be the same for most (if not all) IgG1 antibodies.
a. dissolve 100 mM NHS and subsequently 100 mM EDC in 0.1 M MES buffer pH=5.5
b. incubate the polymer surface with the EDC/NHS solution at RT for 2 hrs
c. gently agitate frequently
d. gently wash the substrate with 1× PBS pH=7.4
e. prepare a 5 μg/ml solution of VU1 D9
f. incubate the polymer surface with the VU1 D9 solution at RT for 2 hrs
g. gently agitate frequently
h. gently wash the substrate with 1× PBS pH=7.4

8. Spaghetti HA Preparation (Comparison)

The previous steps have produced a sample with end-point attached HA. To compare the effect of the end-point attached HA to spaghetti HA, the aminated sample of PU is treated as follows:
a. dissolve 5% w/v 50 kDA HA in 0.1 M MES buffer pH=5.5
b. add 100 mM NHS and subsequently 100 mM EDC gently agitate frequently
c. incubate the polymer surface with the HA solution at RT for 3+ hrs
d. gently agitate frequently
e. gently wash the substrate with 1× PBS pH=7.4 three times or more

9. Cell Suspension Preparation

In order to test the effect of end-point attached hyaluronic acid on the adhesion and rolling of cancer cells in flow, cancer cells have been cultured and flowed along the treated substrate. The MCF7 cell line was used for their robust nature and expression of CD44 and EpCAM. The first step is to prepare a suspension of cells.
a. culture the MCF7 cells to confluency according to the protocol provided by ATCC
b. harvest MCF7 cells with Accutase® according to the protocol provided by Innovative Cell Technologies
c. stain MCF7 cells with CMFDA according to the protocol by ThermoFisher
d. suspend the MCF7 cells in DMEM+10% FBS at a concentration of 20,000 cells/ml

10. Flow Cell

To test the effect of end-point attached hyaluronic acid on the adhesion and rolling of cancer cells in flow, a setup was used that consists of a modified syringe pump, a custom flow cell assembly and epifluorescence microscope. The syringe pump was modified in order to pump the cell suspension through tubes connecting to a channel in the flow cell, where an average velocity of 1 mm/s was maintained, while the epifluorescence microscope captures images of the luminal surface of the treated substrate for 20 minutes.

The requirements of the setup are as followed:
a. a syringe pump:
   i. with two 5-60 ml syringes
   ii. with vertically mounted syringes
   iii. with individually configurable infusion/withdrawal rate for syringes
   iv. with controlled heating to 37° C. for both syringes
   v. with configurable infusion/withdrawal rates between 0.001-128 ml/min
b. a flow cell assembly:
   i. capable of providing an even clamping pressure of 3.2 MPa on an assembly of
      1. a microscopy-slide sized treated substrate 1 mm thickness
      2. a microscopy-slide sized PDMS gasket of 0.5 mm thickness
      3. an Ibidi sticky-Slide I Luer with 0.6 mm channel height (80188)
   ii. with controlled heating to 37° C. of the treated substrate
c. an epifluorescence microscope:
   i. with a 420-490 nm bandpass excitation filter
   ii. with a 520 nm longpass emission filter
   iii. with a 4× objective
   iv. with a camera capable of capturing 1280*960 JPEG images with RGB24 format at 100 ms exposure time at ISO100.

11. Data Analysis

The images as captured in the previous step are analyzed to quantify the effect of the end-point attached hyaluronic acid on the flow- and rolling velocity and immobilization rate. The data analysis is performed as follows:
a. the images are preprocessed in Fiji (an open source image processing package based on ImageJ) with a custom macro whereby
   i. the green channel is isolated
   ii. the ‘Subtract Background” function with rolling ball radius of 50 is used
   iii. a static value is subtracted from all images
b. the images are analyzed with the Trackmate plugin
   i. DoG detector with 20 px diameter and 1.0 threshold
   ii. LAP tracker with 30 pix distance and 50 Y penalty
c. the tracks are exported and processed in MS Excel
   i. all tracks with <5 spots are deleted
   ii. a histogram with 0.25 bin size is made
   iii. the bin sizes are converted to μm/s
d. the histograms are compared to a positive and negative control
e. the histograms are compared to non end-point attached HA

The histogram in FIG. 4 shows the distribution of travel velocities of cells/cell clusters over various substrates. All cells/cell clusters that could not be tracked for at least 5 frames were discarded. Each bar represents the bin a range of velocities, i.e., 0-4.9, 5-9.9, 10-14.9, 15-19.9, 20-24.9, and 25-30 μm/s. Cells/cell clusters that were completely immobilized are included in the 0-4.9 μm/s bin. Cells/cell clusters that were traveling faster than 30 μm/s are not included. The cell suspensions were equal in concentration, meaning that if cell/cell cluster counts are low, the cells generally did not interact with the substrate or adhere to it.

The negative control sample is decorated with —COOH groups that do not interact with the cells or cell clusters and prevent attachment to the substrate, as evidenced by the very low 0-4.9 μm/s bin. The positive control sample is decorated with —NH₂ groups, which are known to adhere cells well. The spaghetti sample is coated with HA that is coupled to the substrate along the length of the molecule, which results in a marginal increase in the rolling and adherent cells/cell clusters. The sample with end-point attached HA shows many more cells/cell clusters that adhere or interact. Because spaghetti HA and end-point attached HA differ only in the method of attachment, the data shows that end-point attachment greatly improves the interaction and adhesion of cells/cell clusters with the substrate.

12. Releasing CTC's

Captured CTCs bound to the coated substrate can be gently released from the substrate through enzymatic degradation of the HA. Unlike classic approaches like trypsinization, this approach minimally affects the viability and phenotype of the CTCs and is therefore ideal for a subsequent analysis that requires unaffected cells. Hyaluronidase from bovine testes is preferred, because it is selective, efficient and economical. Other enzymes that can degrade HA or hyaluronidases from other sources may be used instead. The protocol below describes a simple incubation with a hyaluronidase solution.
a. prepare a solution of 200 U/ml hyaluronidase in 1× PBS pH=7.4
b. prewarm the solution to 37° C.
c. incubate the substrate with CTCs at 37° C. for 5 min
d. harvest the solution to obtain the CTCs in suspension

Viable CTC's with unaffected phenotype can be obtained, which is very difficult to achieve and which is highly desirable for further for further screening of clinically relevant parameters.

1. A coating comprising one or more polysaccharides with binding affinity for bioanalytes, for application on the surface of a medical sampling device, wherein the one or more polysaccharides are end-point attached to the surface of the medical device, and wherein the one or more end-point attached polysaccharides have one or more polysaccharides end-point grafted to the side groups extending from their backbone.

2. The coating of claim 1, wherein in addition to the end-point attached polysaccharides also the polysaccharides that are grafted have one or more polysaccharides end-point grafted to the side groups extending from their backbone.

3. A process for applying a coating comprising one or more polysaccharides with binding affinity for bioanalytes onto the surface of a medical sampling device, wherein the one or more polysaccharides are end-point attached to the surface of the device, comprising a step (A) of aminating the surface, followed by a step (B) of end-point attaching the one or more polysaccharides to the amine groups on the surface, and, either step (C) end-point attaching one or more polysaccharides to side groups extending from the backbone of the polysaccharides that are end-point attached onto the surface followed by a step (D) of blocking any residual functional groups on the surface or step (D) followed by step (C).

4. The process as claimed in claim 3, wherein the surface of the device is aminated by aminolysis, preferably by aminolysis by a diamine, preferably with ethylene diamine or diethylenetriamine.

5. The process of claim 3, wherein the polysaccharides are attached to the amine groups on the surface by reductive amination.

6. The process of claim 3, wherein the one or more polysaccharides that are end-point attached comprise one or more glycosaminoglycans, more preferably hyaluronic acid (HA) or a substituted HA.

7. The process of claim 6, wherein the HA has a molecular weight in the range of 40 kDa to 2 MDA, preferably in the range from 50 kDa to 1.5 MDa.

8. The process of claim 3, wherein the residual amine groups are blocked by reacting with a dialdehyde, preferably glutaraldehyde, followed by reacting with an amino acid, preferably 6-aminocaproic acid, preferably followed with a reduction of imine bonds.

9. The process of claim 3, further comprising a step (E) of grafting receptors and/or ligands onto the one or more polysaccharides.

10. A medical sampling device for capture of bioanalytes, preferably circulating tumor cells, having an aminated surface with end-point attached polysaccharides with binding affinity for bioanalytes, wherein the one or more end-point attached polysaccharides have one or more polysaccharides end-point grafted to the side groups extending from their backbone, and wherein any residual amine groups on the surface are blocked.

11. The medical sampling device of claim 10, having a polymeric surface, preferably composed of a polymer capable of aminolysis, preferably composed of polyurethane or polyester, preferably composed of polyurethane.

12. The medical sampling device of claim 10, wherein the surface is on the inside or outside of the device.

13. A method for capturing bioanalytes, preferably CTC's, from a circulating system using the medical sampling device of claim 10, preferably in the form of a modified guidewire or catheter.

14. A method for releasing captured bioanalytes, preferably CTC's, from a medical sampling device used in the method of claim 13, by subjecting the coating thereof to enzymatic degradation, preferably with a hyaluronidase, more preferably with a hyaluronidase from bovine testes.

15. A method for analysis of bioanalytes, preferably CTC's, comprising the capture of bioanalytes according to the method of claim 13, releasing the captured bioanalytes, and subjecting the bioanalytes to screening of clinically relevant parameters.
